(19) United States
(12) Patent Application Publication
(10) Pub. No.: US 2025/0143867 A1
(43) Pub. Date: May 8, 2025

(54) BIFURCATED ENDOPROSTHESIS SYSTEMS AND METHODS

(71) Applicant: W. L. Gore & Associates, Inc., Newark, DE (US)

(72) Inventors: Garrett A. Johnson, Scottsdale, AZ (US); Martin J. Sector, Phoenix, AZ (US); Kimberley N. Wunder, Phoenix, AZ (US); Anna F. Timbie, Sunnyvale, CA (US); Andrew J. Pierce, Newark, DE (US)

(21) Appl. No.: 18/937,795

(22) Filed: Nov. 5, 2024

Related U.S. Application Data
(60) Provisional application No. 63/716,077, filed on Nov. 4, 2024.
(60) Provisional application No. 63/597,235, filed on Nov. 8, 2023.

(51) Int. Cl. A61F 2/07; A61F 2/06

(52) U.S. Cl. CPC A61F 2/07; A61F 2002/065; A61F 2220/0025; A61F 2250/0037; A61F 2250/0039

(57) ABSTRACT

Various concepts relate to Iliac branch endoprostheses extending for a length between a proximal end and a distal end, the iliac bifurcated endoprosthesis bifurcated from a primary flow channel into two flow channels. Various concepts relate to methods of deploying an endoprosthesis system to treat an aortic aneurysm including deploying an iliac bifurcated endoprosthesis into an ipsilateral leg of a bifurcated main body endoprosthesis, or a contralateral gate of a bifurcated main body endoprosthesis.

### CROSS-REFERENCE TO RELATED APPLICATION

This application claims the benefit of Provisional Application No. 63/716,077, filed Nov. 4, 2024, and also to Provisional Application No. 63/597,235, filed Nov. 8, 2023, which are incorporated herein by reference in their entireties for all purposes.

### BACKGROUND

Endoluminal devices are used in a variety of places in the human body to support various anatomical lumens, such as blood vessels, respiratory ducts, and gastrointestinal ducts, for example. These endoluminal devices are typically deployed at a desired treatment site to treat maladies such as diseases or defects of the afore-mentioned anatomical lumens, including vasculature of a patient. Treatment of vascular maladies involving bifurcated lumens can be more challenging than non-bifurcated treatment sites. For example, the abdominal aorta splits at a primary branch into the common iliac arteries, which branch again at opposing secondary branches into the external iliac and internal iliac arteries (or hypogastric arteries). Treatment of such primary and secondary branch anatomy increases procedure complexity with the addition of modular components and associated delivery systems. For example, to address more challenging branched anatomy, modular endoprostheses have been developed that include a main body endoprosthesis and one or more branch endoprostheses that can be assembled with the main body in vivo.

In the event the anatomy to be treated includes further bifurcations, endoprosthesis systems including additional modular components may be implemented. For example, G. Oderich et al, “Techniques of Endovascular Aortoiliac Repair Using an Iliac Branch Endoprosthesis” Vol. 16, No. 8 Aug. 2017 Supplement to Endovasc. Today, describes treatment of complex multi-branch aneurysms using bilateral femoral access in conjunction with a modular endoprosthesis system (including the Gore® Excluder® Iliac Branch Endoprosthesis).

Iliac branch devices were developed to maintain perfusion of the internal iliac arteries when there is presence of a common iliac artery aneurysm that requires exclusion. Despite their utility, a large portion of patients are excluded from on-label treatment due to a small common iliac artery and/or short total treatment length from lowest renal to internal iliac artery as required by some iliac branch devices. The latter is due to the fact that a contralateral limb is required to bridge the iliac branch device with the trunk-ipsilateral endoprosthesis.

### SUMMARY

Various embodiments address endoprosthesis systems and methods for delivering bifurcated endoprostheses (e.g., iliac bifurcated endoprostheses) for patients with short total treatment length(s). In some examples, the bifurcated endoprostheses can be deployed directly into a contralateral gate of a bifurcated main body endoprosthesis (e.g., such as the GORE® EXCLUDER® AAA Endoprosthesis available from W. L. Gore & Associates, Inc.), eliminating any need to employ a contralateral limb bridging endoprosthesis between the bifurcated endoprosthesis and the bifurcated main body endoprosthesis. Similarly, in some implementations, the bifurcated endoprosthesis can be deployed directly into the ipsilateral limb of a bifurcated main body endoprosthesis (e.g., such as the GORE® EXCLUDER® AAA Endoprosthesis), eliminating any need to employ a contralateral limb bridging endoprosthesis between the bifurcated main body endoprosthesis and the bifurcated endoprosthesis in performing a bilateral procedure (i.e., a procedure in which endoprostheses are placed bilaterally in a patient in association with a AAA system procedure).
According to one Example (“Example 1”), a bifurcated endoprosthesis extends for a length between a proximal end and a distal end, the bifurcated endoprosthesis bifurcating from a primary flow channel into two flow channels. The bifurcated endoprosthesis includes a trunk defining the primary flow channel at the proximal end; a first leg extending from the trunk and defining a gate for receiving a branch endoprosthesis; and a second leg extending from the trunk to the distal end, the gate and the second leg defining the two flow channels, the first leg and the second leg bifurcating from the trunk at a bifurcation region having a diameter that is greater than a diameter of the trunk.
According to another Example (“Example 2”), further to Example 1, the trunk has a substantially uniform diameter, and optionally wherein the substantially uniform diameter is about 16 mm.
According to another Example (“Example 3”), further to Examples 1 or 2, the trunk is configured to be received in a contralateral gate of a main body endoprosthesis.
According to another Example (“Example 4”), further to any preceding Example the trunk has a length of at least 3 cm.
According to another Example (“Example 5”), further to any preceding Example, the first leg defining the gate for receiving the branch endoprosthesis is substantially shorter than the second leg, the gate defined by the first leg optionally defining a diameter of about 8 mm, and optionally having a length of about 2.5 cm.
According to another Example (“Example 6”), further to any preceding Example, wherein the second leg is configured to be anchored in an external iliac artery of a patient, the second leg having a proximal length portion near the bifurcation region that has a diameter proximate the bifurcation region which transitions distally to a larger diameter portion proximate to a position along a length of the second leg corresponding to an end of the first leg defining the gate for receiving the branch endoprosthesis, the larger diameter portion extending to the distal end.
According to another Example (“Example 7”), further to Example 1, the first leg defining the gate for receiving the branch endoprosthesis includes an end stent at a distal end of the first leg and a body stent proximal to the end stent, the trunk includes one or more stents, and the second leg includes one or more stents, and further wherein the end stent and body stent define a relatively lower stent density than the stents of the trunk and second leg.
According to another Example (“Example 8”), further to any preceding Example, the first leg has a first section extending from the bifurcation region, a second section, and an intermediate section between the first section and the second section, the first section having a first diameter that is substantially constant along the first section, the second section having a second diameter that is substantially constant along the second section and having a diameter that is smaller than the first diameter, and the intermediate section tapering in diameter between the first section and the second section.
According to another Example (“Example 9”), further to Example 8, the bifurcated endoprosthesis includes the branch endoprosthesis received in the gate defined by the first leg in a complementary fit, the branch endoprosthesis being engaged with at least the intermediate section of the first leg.
According to another Example (“Example 10”), further to Example 9, the branch endoprosthesis is engaged with the first section, the second section and the intermediate section.
According to another Example (“Example 11”), further to any one of Examples 8 to 10, the first leg is self-expanding.
According to another Example (“Example 12”), further to Example 11, the branch endoprosthesis is balloon expandable.
According to another Example (“Example 13”), further to any one of Examples 8 to 12, the first leg defines a retention shoulder for enhancing retention of the branch endoprosthesis within the first leg.
According to another Example (“Example 14”), a method of deploying an endoprosthesis system to treat an aortic aneurysm includes deploying an iliac bifurcated endoprosthesis into a contralateral gate of a bifurcated main body endoprosthesis.
According to another Example (“Example 15”), further to Example 14, the method includes deploying the main body endoprosthesis in an aorta of a patient, and optionally wherein the main body endoprosthesis is a trunk-ipsilateral abdominal aortic aneurysm (AAA) endoprosthesis configured to repair an abdominal aortic aneurysm (AAA).
According to another Example (“Example 16”), further to Examples 14 or 9, the method further includes cannulating the contralateral gate with a guidewire; advancing the iliac bifurcated endoprosthesis over the guidewire and through an introducer sheath into the contralateral gate; and aligning the iliac bifurcated endoprosthesis to the contralateral gate using a radiopaque marker on the contralateral gate and a radiopaque marker on the iliac bifurcated endoprosthesis.
According to another Example (“Example 17”), further to any one of Examples 14 to 16, wherein the iliac bifurcated endoprosthesis is fully deployed from a proximal end positioned in the contralateral gate to a distal end positioned in the external iliac artery.
According to another Example (“Example 18”), a method of deploying an endoprosthesis system to treat an aortic aneurysm includes deploying an iliac bifurcated endoprosthesis into an ipsilateral leg of a bifurcated main body endoprosthesis.
According to another Example (“Example 19”), further to Example 18, the method further includes advancing a guidewire through the deployed ipsilateral leg of the main body endoprosthesis and reversing the guidewire, to pass in a distal direction and back in the proximal direction down the contralateral gate of the main body endoprosthesis.
According to another Example (“Example 20”), further to Example 19, advancing the guidewire includes using a steerable sheath and/or a snare to reverse the guidewire.
According to another Example (“Example 21”), further to Example 20, wherein the reversed guidewire is advanced out of a patient groin and inserted into an internal iliac gate of the iliac bifurcated endoprosthesis prior to deployment of the iliac bifurcated endoprosthesis.
According to another Example (“Example 22”), further to Example 21, the iliac bifurcated endoprosthesis is advanced over an aortic guidewire running through a leading end and over the reversed guidewire running through the first leg of the undeployed bifurcated endoprosthesis using a delivery system of the iliac bifurcated endoprosthesis.
According to another Example (“Example 23”), further to Example 21, the reversed guidewire is used as a rail guide to advance an appropriate size introducer sheath up and over the main body endoprosthesis from a side that is contralateral to the iliac bifurcated endoprosthesis and into an internal iliac gate of the iliac branch device, and further wherein a second guidewire is advanced through the introducer sheath passing up and over through the main body and into the internal iliac gate in order to cannulate the internal iliac artery.
According to another Example (“Example 24”), further to Example 23, an internal iliac branch endoprosthesis is delivered up and over the main body endoprosthesis through the introducer sheath and into the internal iliac gate of the iliac bifurcated endoprosthesis and into the internal iliac artery, and further wherein the internal iliac branch endoprosthesis is deployed in the internal iliac artery and the internal iliac gate of the iliac bifurcated endoprosthesis.
According to another Example (“Example 25”), a bifurcated endoprosthesis extending for a length between a proximal end and a distal end bifurcates from a primary flow channel into two flow channels and includes a trunk defining the primary flow channel at the proximal end; a first leg extending from the trunk and defining a gate for receiving a branch endoprosthesis; and a second leg extending from the trunk to the distal end, the first leg and the second leg defining the two flow channels extending from the primary flow channel, the first leg and the second leg bifurcating from the trunk at a bifurcation region, the first leg having a first section extending from the bifurcation region, a second section, and an intermediate section between the first section and the second section, the first section having a first diameter that is substantially constant along the first section, the second section having a second diameter that is substantially constant along the second section and having a diameter that is smaller than the first diameter, and the intermediate section tapering in diameter between the first section and the second section.
According to another Example (“Example 26”), further to Example 25, the bifurcated endoprosthesis further includes the branch endoprosthesis received in the gate defined by the first leg in a complementary fit, the branch endoprosthesis being engaged with at least the intermediate section of the first leg.
According to another Example (“Example 27”), further to Example 26, the branch endoprosthesis is engaged with the first section, the second section and the intermediate section.
According to another Example (“Example 28”), further to any one of Examples 25 to 27, wherein the first leg is self-expanding.
According to another Example (“Example 26”), further to Example 28, the branch endoprosthesis is balloon expandable.
According to another Example (“Example 30”), further to any one of Examples 25 to 29, the first leg defines a retention shoulder for enhancing retention of the branch endoprosthesis within the first leg.
According to another Example (“Example 31”), further to any one of Examples 25 to 30, wherein the branch endoprosthesis is an internal iliac branch endoprosthesis.
According to another Example (“Example 32”), further to any one of Examples 25 to 31, configured as an iliac bifurcated endoprosthesis.
According to another Example (“Example 33”), a method of deploying an endoprosthesis system to treat an aortic aneurysm includes deploying an iliac bifurcated endoprosthesis within a contralateral gate of a main body endoprosthesis; forming a complementary fit between a retention shoulder of a first leg of the iliac bifurcated endoprosthesis with an internal iliac branch endoprosthesis received in the first leg.
According to another Example (“Example 34”), further to Example 33, the internal iliac branch endoprosthesis includes a balloon expandable stent and forming the complementary fit includes balloon expanding the internal iliac branch endoprosthesis.
According to another Example (“Example 35”), further to Examples 33 or 34, wherein the first leg has a first section, a second section, and an intermediate section between the first section and the second section, the first section having a first diameter that is substantially constant along the first section, the second section having a second diameter that is substantially constant along the second section and having a diameter that is smaller than the first diameter, and the intermediate section tapering in diameter between the first section and the second section, wherein the intermediate section defines the retention shoulder of the first leg and forming the complementary fit includes engaging the internal iliac branch endoprosthesis with the first section, the intermediate section and the second section.

The various examples provided in this patent specification should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 shows a bifurcated endoprosthesis, according to some embodiments.

FIGS. 2 to 7 show a deployment method for an endoprosthesis system for deployment in the abdominal aorta, according to some embodiments.

FIGS. 8 to 11 show another deployment method for an endoprosthesis system for deployment in the abdominal aorta, according to some embodiments.

FIGS. 12A to 14 show additional features of a bifurcated endoprosthesis, according to some embodiments.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The various embodiment methods and devices provided by this description, including the associated figures, may be used in association with vascular treatment methods, such as treatment of an aortic aneurysm (abdominal aortic aneurysm (AAA). Although the specific application to aortic aneurysms is described, other treatments using the designs and associated treatment methods described herein are contemplated.

### Branch Endoprosthesis Designs

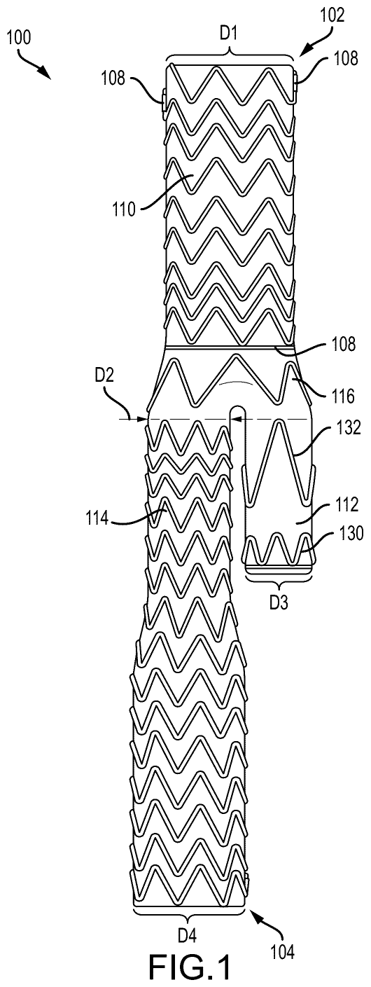

FIG. 1 shows a bifurcated endoprosthesis 100 (e.g., an iliac bifurcated endoprosthesis), according to some embodiments. The bifurcated endoprosthesis 100 is formed of one or more graft components and one or more frame or stent components. In various examples, the bifurcated endoprosthesis 100 is self-expanding in design, the bifurcated endoprosthesis including one or more self-expanding stent components. The bifurcated endoprosthesis 100 may be formed of similar materials and be similarly configured to be self-expanding as the GORE® EXCLUDER® Iliac Branch Endoprosthesis, according to some embodiments.

As shown, the bifurcated endoprosthesis 100 is bifurcated from a primary flow channel into two bifurcated flow channels. The bifurcated endoprosthesis 100 extends for a length between a proximal end 102 and a distal end 104, the bifurcated endoprosthesis 100 including a trunk 110 defining the primary flow channel at the proximal end 102 and a first leg 112 extending from the trunk 110 and a second end 114 extending from the trunk to the distal end 104, the two legs 112, 114 defining the two bifurcated flow channels, with the two legs 112, 114 bifurcating from the trunk 110 at a bifurcation region 116.

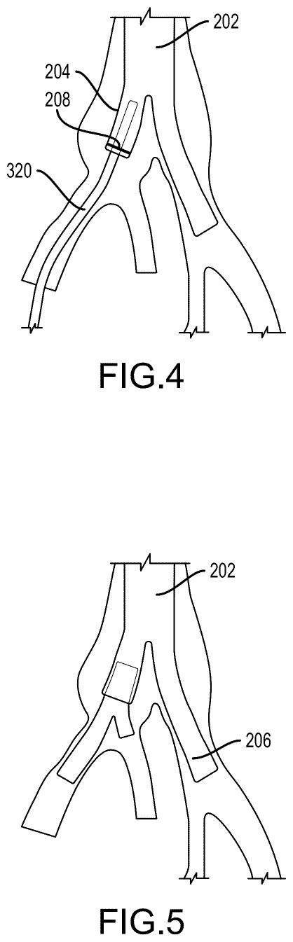

As shown, the trunk 110 has a substantially uniform diameter D1 (e.g., about 16 mm) although a variety of shapes, sizes, and dimensions are contemplated. Generally, the trunk 110 is sized, shaped, and otherwise configured to be received within a main body endoprosthesis (e.g., FIG. 3), such as the GORE® EXCLUDER® AAA Endoprosthesis. In some embodiments, the trunk 110 has a length of about 4 cm, or at least 3 cm, in order to promote sufficient overlap for sealing and coupling to the main body endoprosthesis, although a variety of dimensions are contemplated.

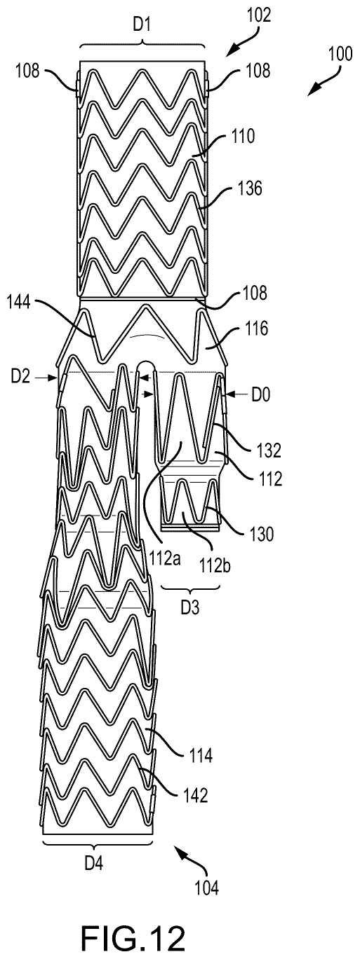

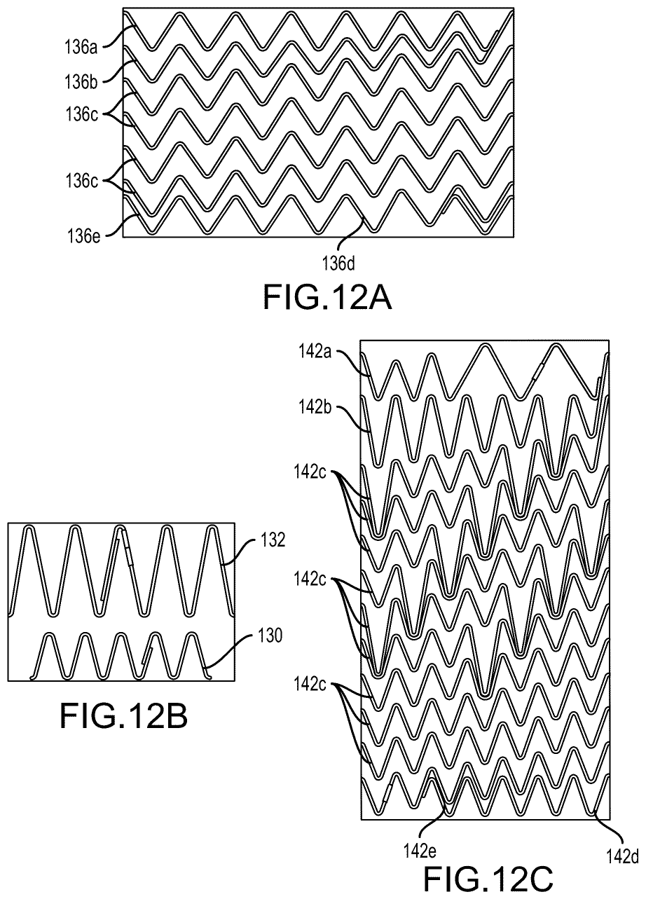

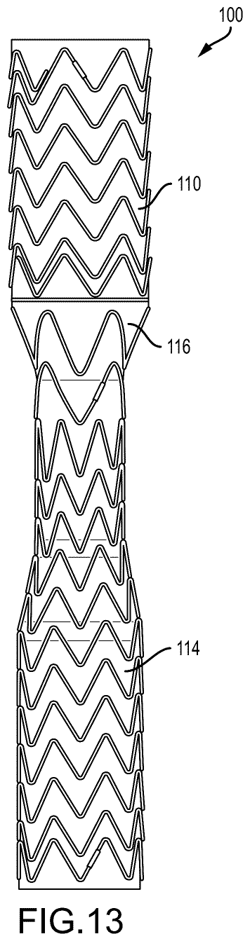

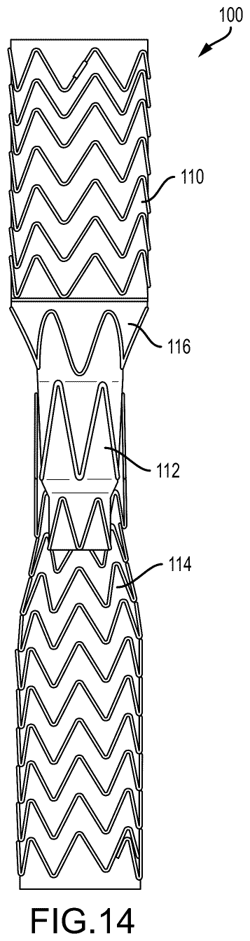

As shown, the first leg 112 is substantially shorter than the second leg 114, the first leg serving as a gate, or coupling feature for a branch endoprosthesis 250 (e.g., an internal iliac branch endoprosthesis). The first leg 112 defines a diameter D3 of about 8 mm, and in some examples has a length of about 1.5 cm, although a variety of dimensions are contemplated. As subsequently described, in some embodiments (FIGS. 12 to 14), the first leg 112 includes a tapered profile which can help facilitate enhanced retention capabilities. As one example, the first leg 112 may define a starting diameter D0 of 9 mm proximate the bifurcation region 116 and taper to a diameter D3 of about 7 mm, with a length of about 2 cm, although a variety of dimensions are contemplated and the foregoing dimensions are not meant to limit all tapered embodiments.

The second leg 114 is optionally configured to be placed in and anchored in a vessel, and may serves as an external iliac leg for being placed and anchored in an external iliac artery. For example, the second leg 114 has a portion with a proximal length near, or proximate to the bifurcation region 116 that has a diameter D2 of 10 mm and may transition distally to a portion having a larger diameter D4 located proximate to an end of the first leg 112 and extending to the distal end 104. In some embodiments, D4 is about 10 mm, 12 mm, 14.5 mm, or other dimension as desired to facilitate securement in the external iliac artery. The proximal (smaller diameter) portion of the second leg 114 may have a length of about 20 mm, for example, and the distal (larger diameter) portion of the second leg 114 may have a length of about 30 mm, for example. The tapered region between the two portions may have a length of about 10 mm, for example. Although some specific dimensional values have been provided, these are provided by way of example, and not all embodiments of the second leg 114 should be limited to those example values.

As shown, the bifurcation region 116 itself (e.g., having an increasing taper in diameter, or increasing in diameter, from the trunk 110 to the first and second legs 112, 114) may have a greater diameter than the trunk 110.

In some embodiments, the stent component(s) of the first leg 112 may include an end stent 130 and a body stent 132 proximal to the end stent 130. As shown, the end stent 130 and body stent 132 may define a relatively lower stent density (e.g., less stent material over length) than the stent components of the trunk 110 and second leg 114. In various examples, lower stent density is sufficient in the first leg 112 due to the use of a balloon expandable (vs. self-expanding) design for the branch endoprosthesis 250 (e.g., internal iliac branch endoprosthesis) secured within the first leg 112.

As shown in FIGS. 12 to 14, some embodiments of the bifurcated endoprosthesis 100 may be configured to further enhance or promote greater engagement and securement within the first leg 112, and thus less stent reinforcement may be required in the first leg 112, according to some embodiments. In particular, FIGS. 12 to 14 illustrate the bifurcated endoprosthesis 100 with the first leg 112 having a modified, tapered configuration relative to the design shown in FIG. 1. Additionally, the trunk 110, the first leg 112, and the second leg 114 each have modified frame patterns, which are also described in greater detail below.

As shown in FIGS. 12 to 14, the overall shape of the trunk 110 is cylindrical and similar to the design of FIG. 1. The trunk 110 may have a nominal diameter of about 16 mm and a length of about 30 mm, although a variety of dimensions are contemplated and not all embodiments of the trunk 110 should be limited to those examples. The second leg 114 is substantially similar to that shown in FIG. 1, and is left from further description apart from the stent, or frame pattern, which is discussed in more detail below. The first leg 112 has a modified overall shape relative to that shown in FIG. 1, but similar to the embodiments described above the first leg 112 serves as a gate, or coupling feature for the branch endoprosthesis 250 (e.g., internal iliac branch endoprosthesis). Although some specific dimensional values have been provided, these are provided by way of example, and not all embodiments of the trunk 110 should be limited to those example values.

As shown, the first leg 112 has a first section 112a extending from the bifurcation region 116, a second section 112b extending generally in a direction of the distal end 104, and an intermediate section 112c between the first section 112a and the second section 112b. The first section 112a has a first diameter Da that is substantially constant along the first section 112a. The second section 112b has a second diameter Db that is substantially constant along the second section 112b. As shown, the second diameter Db is smaller than the first diameter Da. The intermediate section 112c tapers in diameter between the first section 112a and the second section 112b. The intermediate section 112c can help define a retention shoulder, or retention feature, for enhancing retention of the branch endoprosthesis 250. In particular, the branch endoprosthesis 250 may be expanded (e.g., balloon expanded) to form a complementary fit with the at least the intermediate section 112c and the second section 112b, and preferably with the first section 112a, the second section 112b, and the intermediate section 112c.

The first diameter Da of the first section 112a may be about 10% greater than second section 112b, about 20% greater than the second section, about 30% greater than the second section, or any value or range between any of the foregoing values. In some embodiments, the first diameter Da of the first section 112a may be about 9 mm. The first diameter Da may be an inner diameter of the first section 112a. In some embodiments, the second diameter Db of the second section 112b may be about 7 mm. The second diameter Db may be an inner diameter of the second section 112b. The length of the first leg 112 may be about 20 mm. Although some specific dimensional values have been provided, these are provided by way of example, and not all embodiments of the first leg 112 should be limited to those example values. In some embodiments, the first section 112a and the second section 112b are supported by one or more stents, and the intermediate section 112c is unsupported by a stent. Although some specific dimensional values have been provided, these are provided by way of example, and not all embodiments of the first leg 112 should be limited to those example values.

As shown in FIGS. 12 to 14, the trunk 110 has a stent 136 (e.g., a self-expanding, helically wound stent). FIG. 12A is a flattened schematic view of a potential wind pattern for the stent 136. As shown, the stent 136 has a proximal end row 136a, a proximal transition row 136b, a plurality of body rows 136c, a distal transition row 136d, and a distal end row 136e. In some examples, the stent 136 has a wire diameter of about 0.01 inches. The stent 136 may have about 7 apices per row, with apices having about a 0.040 inch radius of curvature, a pitch between apices of adjacent rows of about 0.18 inches (4.5 mm), an amplitude of about 5 mm, and an interlock, or overlap between rows of about 0.6 mm. The distal transition row 136d may be a partial row (e.g., less than a full turn or revolution). As shown, the end rows (the proximal most and distal most rows) are generally square, or at a right angle relative to a longitudinal axis of the trunk 110. Although some specific dimensional values have been provided, these are provided by way of example, and not all embodiments of the trunk 110 should be limited to those example values.

As shown in FIGS. 12 to 14, the first leg 112 has an end stent 130 (e.g., corresponding to the second section 112b) and a body stent 132 (e.g., corresponding to the first section 112a). FIG. 12B is a flattened schematic view of a potential wind pattern for the end stent 130 and the body stent 132. The wire diameter of the body stent 132 and the end stent 130 may be about 0.01 inches. As shown, each of the end stent 130 and the body stent 132 may have about 5 apices. The end stent 130 may have an amplitude of about 6 mm and the body stent 132 may have an amplitude of about 12 mm. The apices of the end stent 130 and the body stent 132 may have a radius of curvature of about 0.02 inches, for example. In various examples, the relatively shorter stent struts and overall amplitude (e.g., being about 50% or less) may result in the end stent 130, and thus the second section 112b, having greater hoop strength or resistance to expansion. In some examples, the end stent 130 and the body stent 132 do not overlap, or interlock, and have about a 2 mm gap between them. Although some specific dimensional values have been provided, these are provided by way of example, and not all embodiments of the first leg 112 should be limited to those example values.

As shown in FIGS. 12 to 14, the second leg 114 has a stent 142 (e.g., a self-expanding, helically wound stent). FIG. 12C is a flattened schematic view of a potential wind pattern for the stent 142, including a proximal end row 142a, a proximal transition row 142b, a plurality of body rows 142c, a distal transition row 142d, and a distal end row 142e. In some examples, the stent 142 has a wire diameter of about 0.01 inches. The stent 136 may have about 7 apices per row, with apices having about a 0.020 inch radius of curvature, a pitch between apices of adjacent rows of about 0.18 inches (4.5 mm), an amplitude of about 5 mm. The proximal end row 142a may define an interlock, or overlap with the stent 144 of the bifurcation region 116. As shown, the proximal transition row 142b may have at least one apex that defines a substantial interlock (a global, or complete overlap) with one of the plurality of body rows 142a, and each of the body rows 142c may have at least one apex that defines a substantial interlock (a global, or complete overlap) with an adjacent one of the plurality of body rows 142a. The distal transition row 142d may be a partial row (e.g., less than a full turn or revolution). The most distal of the plurality of body rows 142c may interlock, or overlap with the distal end row 142e by about 3 mm, for example. As shown, the end rows (the proximal most and distal most rows) corresponding to the proximal end row 142a and the distal end row 142e are generally square, or at a right angle relative to a longitudinal axis of the second leg 114. Although some specific dimensional values have been provided, these are provided by way of example, and not all embodiments of the second leg 114 should be limited to those example values.

As shown in FIGS. 12 to 14, the bifurcation region 116 has a stent 144 (e.g., a self-expanding, helically wound stent or a non-helical, ring stent). The stent 144 may have a wire diameter of about 0.01 inches. The stent 144 may have about 7 apices and a single row, for example. Although some specific dimensional values have been provided, these are provided by way of example, and not all embodiments of the bifurcation region 116 should be limited to those example values.

### Methods of Delivery

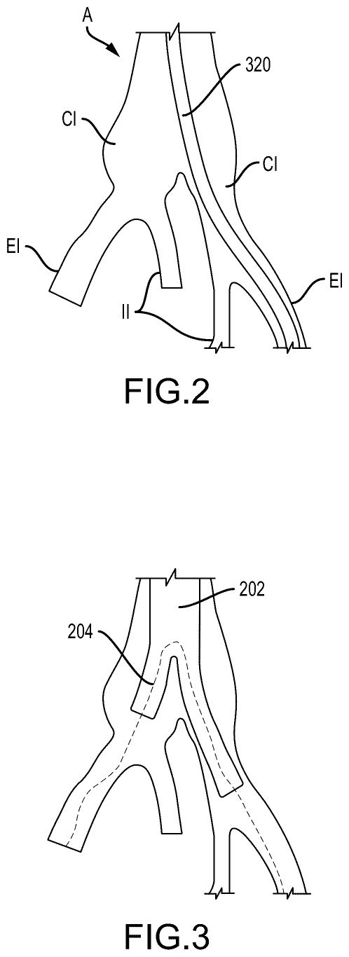

FIGS. 2 to 7 illustrate a method of deploying an endoprosthesis system for treating an abdominal aorta A of a patient including deploying the bifurcated endoprosthesis 100 into a bifurcated main body endoprosthesis 202 (FIG. 3), and in particular into a contralateral gate 204 of the bifurcated main body endoprosthesis 202.

As shown in FIGS. 2 and 3, according to some methods, after the patient is prepared per standard surgical practices (from initial arterial access to guidewire visualization at the target vessels), the main body endoprosthesis 202 (e.g., trunk-ipsilateral AAA endoprosthesis) is deployed to the contralateral gate 204 using a main body delivery system 310. The ipsilateral leg 206 of the main body endoprosthesis 202 is then deployed.

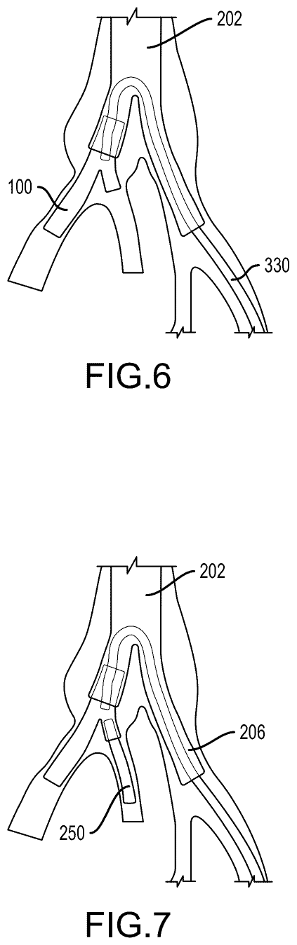

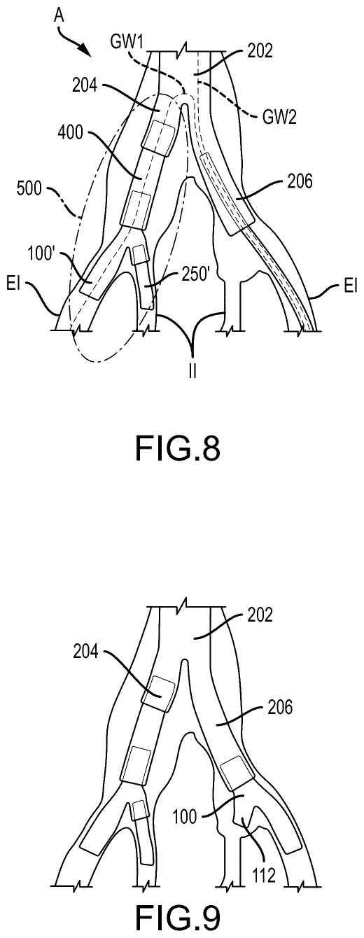

As shown in FIGS. 4 and 5, once the contralateral gate 204 is cannulated with a guidewire (e.g., not shown, but optionally deployed in a similar manner to GW1 shown in FIG. 8), the bifurcated endoprosthesis 100 (an iliac bifurcated endoprosthesis as shown) is advanced over the guidewire and through an introducer sheath using an iliac bifurcated endoprosthesis delivery system 320 into the contralateral gate 204. The guidewire may be reversed, or curved, extending up from the external iliac, for example, through the main body endoprosthesis 202 ipsilateral leg 206 and back down through the ipsilateral leg 206, and out from the opposite external iliac (e.g., again, not shown, but optionally deployed in a similar manner to GW1 shown in FIG. 8). The guidewire may be accessible from two access sites on either side of the patient, for example. The guidewire delivery and reversal procedure may be accomplished according to known methods, including use of a steering catheter and/or a guidewire snare. The undeployed bifurcated endoprosthesis 100, and in particular the first leg 112, may be pre-cannulated with a removable guidewire tube to facilitate introduction of a guidewire through the first leg 112. In some embodiments, the reversed guidewire is advanced out of the patient groin and inserted into the pre-cannulated first leg 112 (e.g., serving as an internal iliac gate) of the undeployed bifurcated endoprosthesis 100. The bifurcated endoprosthesis 100 is then advanced over an aortic guidewire running through the leading end of a delivery system of the iliac bifurcated endoprosthesis 100 and over the reversed guidewire running through the first leg 112 of the undeployed bifurcated endoprosthesis 100. In some methods, the bifurcated endoprosthesis 100 is aligned to the main body endoprosthesis 202 using a radiopaque marker 208 (an example of which is shown on FIG. 4 for visualization purposes) on the main body endoprosthesis 202 contralateral gate 204 which can be aligned to one or more radiopaque markers 108 (examples of which are shown on the designs of FIGS. 1 and 12) on a leading end (e.g., the proximal end 102) of the bifurcated endoprosthesis 100 or otherwise positioned on the trunk 110. The bifurcated endoprosthesis 100 is then fully deployed from the proximal end 102 positioned in the contralateral gate 204 to the distal end 104 positioned in the external iliac artery El.

As shown in FIGS. 6 and 7, after the delivery system is withdrawn, and according to some embodiments, the guidewire (not shown) is reversed in direction, to pass in one direction in the main body endoprosthesis 202 from an ipsilateral leg 206 back into the contralateral gate 204, in a second direction through the first leg 112 (internal iliac gate) of the deployed bifurcated endoprosthesis 100 (e.g., in a distal direction, and then back in a proximal direction as defined relative to the access point), in a guide reversal step. As subsequently described, various embodiments include use of a guide reversal system (e.g., including a steerable sheath and/or a snare) to achieve reversal of the guidewire. The reversed guidewire is then advanced into the internal iliac artery II. An appropriately sized introducer sheath (e.g., similar to introducer sheath 600 shown in FIG. 11) is then advanced over the reversed guidewire and into the internal iliac artery II. The branch endoprosthesis 250 (e.g., an internal iliac branch endoprosthesis) is advanced using a branch delivery system 330 (e.g., an internal iliac branch delivery system) through the introducer sheath and into the internal iliac artery II. The branch endoprosthesis 250 is aligned using suitable radiopaque markers on the first leg 112 (internal iliac gate) of the bifurcated endoprosthesis 100.

In some examples (e.g., those including use of a design such as that represented in FIG. 12), the method includes forming a complementary fit between the retention shoulder defined by the intermediate section 112c of a first leg 112 of the bifurcated endoprosthesis 100 and the branch endoprosthesis 250 received in the first leg 112. The method may include balloon expanding the branch endoprosthesis 250 to form the complementary fit such that the branch endoprosthesis 250 is engaged with the first leg 112. For example, the branch endoprosthesis 250 may include a balloon expandable stent (plastically deformable) and forming the complementary fit includes balloon expanding the branch endoprosthesis 250 to engage with the first leg (e.g., the retention shoulder).

After the branch endoprosthesis 250 is deployed and associated delivery system 330 withdrawn, the proximal end of the branch endoprosthesis 250 is ballooned in the first leg 112 (internal iliac gate) with an appropriate size balloon. A distal end of the branch endoprosthesis 250 in the internal iliac artery II is then ballooned with an appropriate size balloon. The reversed guidewire is then withdrawn from the internal iliac artery II and back through the first leg 112 (internal iliac gate) of the bifurcated endoprosthesis 100. In some methods, the guidewire is then used to balloon the proximal end 102 of the bifurcated endoprosthesis 100 that is deployed in the contralateral gate 204 of the main body endoprosthesis 202 as well as the distal end 104 that is deployed in the external iliac artery El. Additional procedural steps may be taken as per standard surgical practices.

FIGS. 8 to 11 illustrate another method of deploying an endoprosthesis system for treating an abdominal aorta A of a patient including deploying the bifurcated endoprosthesis 100 into the bifurcated main body endoprosthesis 202, and in particular into the ipsilateral leg 206 of the bifurcated main body endoprosthesis 202.

As shown in FIG. 8, after the patient is prepared per standard surgical practices (e.g., from initial arterial access to guidewire visualization at the target vessels), the main body endoprosthesis 202 is deployed up to the contralateral gate 204. Once the contralateral gate 204 is cannulated with a guidewire, the main body endoprosthesis 202 is deployed along with the deployed assembly 500 on the side of the contralateral gate 204, which includes a bifurcated endoprosthesis 100′ (e.g., an iliac bifurcated endoprosthesis), branch endoprosthesis 250′ (internal iliac branch endoprosthesis), and a contralateral extension endoprosthesis 400.

Once the deployed assembly 500 is in place, a guidewire GW1 (e.g., FIG. 8) is advanced through the deployed ipsilateral leg 206 of the main body endoprosthesis 202 and reversed in direction, to pass in one direction in the main body endoprosthesis 202 and back, in a second direction down the contralateral gate 204 of the main body endoprosthesis 202 (e.g., in a distal direction, and then back in a proximal direction as defined relative to the access point), in a guide reversal step. Various embodiments include use of a guide reversal system (e.g., including a steerable sheath and/or a snare) to achieve reversal of the guidewire. For reference the guidewire GW1 is illustrated in FIG. 8 and left from the remaining figures for ease of visualization of the remaining parts.

The undeployed bifurcated endoprosthesis 100, and in particular the first leg 112, may be pre-cannulated with a removable guidewire tube (not shown) to facilitate introduction of a guidewire GW1 through the first leg 112. In some embodiments, the reversed guidewire is advanced out of the patient groin and inserted into the removable guidewire tube pre-cannulated in the first leg 112 (internal iliac gate) of the undeployed bifurcated endoprosthesis 100. The bifurcated endoprosthesis 100 is then advanced over an aortic guidewire GW2 (e.g., FIG. 8) running through the leading end of a delivery system of the iliac bifurcated endoprosthesis 100 and over the reversed guidewire GW1 running through the first leg 112 of the undeployed branch endoprosthesis 100.

The bifurcated endoprosthesis 100 is advanced through an introducer sheath, and into the ipsilateral leg 206 of the main body endoprosthesis 202. The bifurcated endoprosthesis 100 is optionally positioned using one or more radiopaque markers on the main body endoprosthesis 202 that are aligned with radiopaque markers on the leading end of the bifurcated endoprosthesis 100. The bifurcated endoprosthesis 100 is then deployed (expanded) in a direction of deployment from the proximal end 102 positioned in the ipsilateral leg 206 to the first leg 112 of the bifurcated endoprosthesis 100.

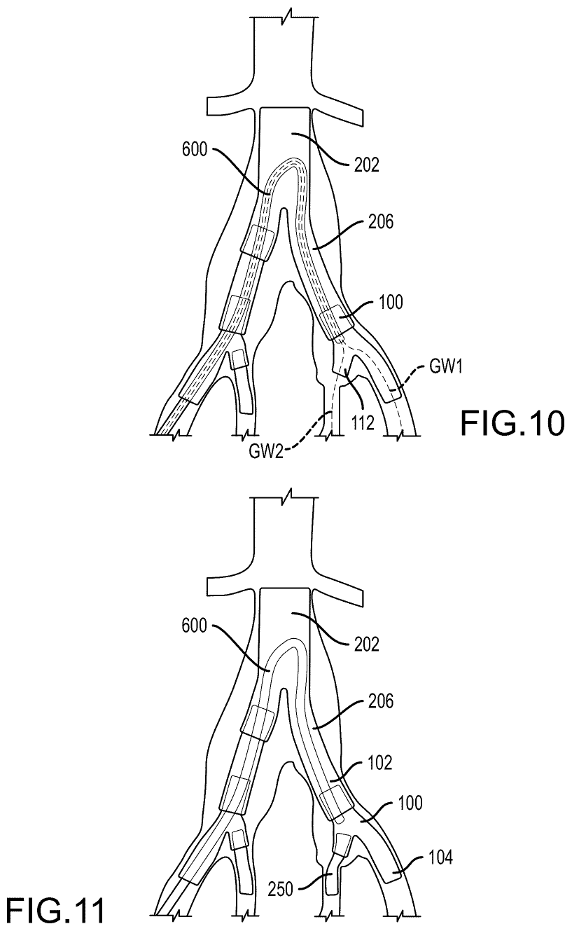

As shown in FIG. 10, in some embodiments, the reversed guidewire GW1 can be used as a rail guide to advance an appropriate size introducer sheath 600 up and over the main body endoprosthesis 202 from the side contralateral to the bifurcated endoprosthesis 100 and into the first leg 112 of the bifurcated endoprosthesis 100. A second guidewire GW2 is advanced through the introducer sheath passing up and over through the main body 102 and into the first leg 112 in order to cannulate the internal iliac artery II.

The branch endoprosthesis 250 (internal iliac branch endoprosthesis) is then delivered up and over the main body endoprosthesis 202 through the up and over introducer sheath and into the first leg 112 and internal iliac artery II. In some embodiments, the branch endoprosthesis 250 is properly aligned in the first leg 112 using a radiopaque marker on the first leg 112 of the bifurcated endoprosthesis 100. After the branch endoprosthesis 250 is deployed and associated delivery system withdrawn, the proximal end of the branch endoprosthesis 250 is ballooned in the first leg 112 with an appropriate size balloon. The distal end of the branch endoprosthesis 250 that is positioned in the internal iliac artery II is then ballooned/expanded with an appropriate size balloon. The reversed guidewire GW2 is then withdrawn from the internal iliac artery II and back through the first leg 112 of the bifurcated endoprosthesis 100. The guidewire GW1 is then used to direct a balloon catheter to balloon the proximal end 102 of the bifurcated endoprosthesis 100 that is deployed in the ipsilateral leg 206 of the main body endoprosthesis 202 as well as the distal end 104 deployed in the external iliac artery El. The procedure is then completed as per standard surgical practices.

The methodology described above in association with FIGS. 2 to 7 and/or 8 to 11 can be employed when bilateral iliac branch devices (e.g., two branch endoprostheses 100) are required and total treatment length of the ipsilateral leg 206 side of the main body endoprosthesis 202 is shorter than can accommodate all deployed components. The bifurcated endoprosthesis 100 can be deployed similarly to the manner described in association with FIGS. 2 to 7 or FIGS. 8 to 11 above. In such an example, the bifurcated endoprosthesis 100 is deployed with the branch endoprosthesis 250 (internal iliac branch endoprosthesis) being delivered up and over the bifurcated endoprosthesis 100, but prior to complete deployment of the main body endoprosthesis 202. Alternatively, in a manner similar to that described in association with FIGS. 8 to 11, the bifurcated endoprosthesis 100 can be deployed in a manner in which the branch endoprosthesis 250 is delivered up and over the main body endoprosthesis 202 after the main body endoprosthesis 202 is completely deployed.

### Materials

The materials used for the graft components associated with the various endoprostheses can include any material which is suitable for use as a graft in the chosen body lumen. The graft components for the various endoprostheses can be composed of the same or different materials between the various endoprostheses. The graft components can comprise multiple layers of material that can be the same material or different materials. The graft components may have a layer that is formed into a tube (innermost tube) and an outermost layer that is formed into a tube (outermost tube).

Many graft materials are known, particularly known are those that can be used as vascular graft materials. The graft materials can be extruded, coated or formed from wrapped films, or a combination thereof.

Polymers, biodegradable and natural materials can be used for specific applications. Biocompatible materials in particular are contemplated for the various graft components. In certain instances, the graft components may include a fluoropolymer, such as a polytetrafluoroethylene (PTFE) polymer or an expanded polytetrafluoroethylene (ePTFE) polymer. In some instances, the graft components may be formed of, such as, but not limited to, a polyester, a silicone, a urethane, a polyethylene terephthalate, or another biocompatible polymer, or combinations thereof. In some instances, bioresorbable or bioabsorbable materials may be used, for example a bioresorbable or bioabsorbable polymer. In some instances, the graft can include Dacron, polyolefins, carboxy methylcellulose fabrics, polyurethanes, or other woven, non-woven, or film elastomers.

Biocompatible materials may be used for the various frame components, or stent components, associated with the endoprostheses described herein. For example, nitinol (NiTi) may be used as the material of the frame or stent (and any of the frames discussed herein), but other materials such as, but not limited to, stainless steel, L605 steel, polymers, MP35N steel, polymeric materials, Pyhnox, Elgiloy, or any other appropriate biocompatible material, and combinations thereof, can be used as the material of the frame. The super-elastic properties and softness of NiTi may enhance the conformability of the stent. In addition, NiTi can be shape-set into a desired shape. That is, NiTi can be shape-set so that the frame tends to self-expand into a desired shape when the frame is unconstrained, such as when the frame is deployed out from a delivery system. Self-expanding stent component materials and balloon expandable stent component materials are contemplated.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A bifurcated endoprosthesis extending for a length between a proximal end and a distal end, the bifurcated endoprosthesis bifurcating from a primary flow channel into two flow channels, the bifurcated endoprosthesis comprising:
a trunk defining the primary flow channel at the proximal end;
a first leg extending from the trunk and defining a gate for receiving a branch endoprosthesis; and
a second leg extending from the trunk to the distal end, the gate and the second leg defining the two flow channels, the first leg and the second leg bifurcating from the trunk at a bifurcation region having a diameter that is greater than a diameter of the trunk.

2. The bifurcated endoprosthesis of claim 1, wherein the trunk has a substantially uniform diameter, and optionally wherein the substantially uniform diameter is about 16 mm.

3. The bifurcated endoprosthesis of claim 1, wherein the trunk is configured to be received in a contralateral gate of a main body endoprosthesis.

4. The bifurcated endoprosthesis of claim 1, wherein the trunk has a length of at least 3 cm.

5. The bifurcated endoprosthesis of claim 1, wherein the first leg defining the gate for receiving the branch endoprosthesis is substantially shorter than the second leg, the gate defined by the first leg optionally defining a diameter of about 8 mm, and optionally having a length of about 2.5 cm.

6. The bifurcated endoprosthesis of claim 1, wherein the second leg is configured to be anchored in an external iliac artery of a patient, the second leg having a proximal length portion near the bifurcation region that has a diameter proximate the bifurcation region which transitions distally to a larger diameter portion proximate to a position along a length of the second leg corresponding to an end of the first leg defining the gate for receiving the branch endoprosthesis, the larger diameter portion extending to the distal end.

7. The bifurcated endoprosthesis of claim 1, wherein the first leg defining the gate for receiving the branch endoprosthesis includes an end stent at a distal end of the first leg and a body stent proximal to the end stent, the trunk includes one or more stents, and the second leg includes one or more stents, and further wherein the end stent and body stent define a relatively lower stent density than the stents of the trunk and second leg.

8. The bifurcated endoprosthesis of claim 1, wherein the first leg has a first section extending from the bifurcation region, a second section, and an intermediate section between the first section and the second section, the first section having a first diameter that is substantially constant along the first section, the second section having a second diameter that is substantially constant along the second section and having a diameter that is smaller than the first diameter, and the intermediate section tapering in diameter between the first section and the second section.

9. The bifurcated endoprosthesis of claim 8, further comprising the branch endoprosthesis received in the gate defined by the first leg in a complementary fit, the branch endoprosthesis being engaged with at least the intermediate section of the first leg.

10. The bifurcated endoprosthesis of claim 9, wherein the branch endoprosthesis is engaged with the first section, the second section and the intermediate section.

11. The bifurcated endoprosthesis of claim 8, wherein the first leg is self-expanding.

12. The bifurcated endoprosthesis of claim 11, wherein the branch endoprosthesis is balloon expandable.

13. The bifurcated endoprosthesis of claim 8, wherein the first leg defines a retention shoulder for enhancing retention of the branch endoprosthesis within the first leg.

14. A method of deploying an endoprosthesis system to treat an aortic aneurysm, the method comprising:
deploying an iliac bifurcated endoprosthesis into a contralateral gate of a bifurcated main body endoprosthesis.

15. A method of deploying an endoprosthesis system to treat an aortic aneurysm, the method comprising:
deploying an iliac bifurcated endoprosthesis into an ipsilateral leg of a bifurcated main body endoprosthesis.

16. A bifurcated endoprosthesis extending for a length between a proximal end and a distal end, the bifurcated endoprosthesis bifurcating from a primary flow channel into two flow channels, the endoprosthesis comprising:
a trunk defining the primary flow channel at the proximal end;
a first leg extending from the trunk and defining a gate for receiving a branch endoprosthesis; and
a second leg extending from the trunk to the distal end, the first leg and the second leg defining the two flow channels extending from the primary flow channel, the first leg and the second leg bifurcating from the trunk at a bifurcation region, the first leg having a first section extending from the bifurcation region, a second section, and an intermediate section between the first section and the second section, the first section having a first diameter that is substantially constant along the first section, the second section having a second diameter that is substantially constant along the second section and having a diameter that is smaller than the first diameter, and the intermediate section tapering in diameter between the first section and the second section.

17. The bifurcated endoprosthesis of claim 16, further comprising the branch endoprosthesis received in the gate defined by the first leg in a complementary fit, the branch endoprosthesis being engaged with at least the intermediate section of the first leg.

18. The bifurcated endoprosthesis of claim 17, wherein the branch endoprosthesis is engaged with the first section, the second section and the intermediate section.

19. The bifurcated endoprosthesis of claim 16, wherein the first leg is self-expanding.

20. The bifurcated endoprosthesis of claim 19, wherein the branch endoprosthesis is balloon expandable.

21. The bifurcated endoprosthesis of claim 16, wherein the first leg defines a retention shoulder for enhancing retention of the branch endoprosthesis within the first leg.

22. The bifurcated endoprosthesis of claim 16, wherein the branch endoprosthesis is an internal iliac branch endoprosthesis.

23. The bifurcated endoprosthesis of claim 16 configured as an iliac bifurcated endoprosthesis.
